(19) United States
(12) Patent Application Publication
(10) Pub. No.: US 2009/0326618 A1
(43) Pub. Date: Dec. 31, 2009

(54) Intense Pulsed Light Device

(76) Inventor: Geoffrey Clive Davies, Swansea (GB)

Correspondence Address: KING & SCHICKLI, PLLC, 247 NORTH BROADWAY, LEXINGTON, KY, 40507, US

(21) Appl. No.: 12/374,760

(22) PCT Filed: Jul. 24, 2007
(86) PCT No.: PCT/GB2007/002803

§ 371 Date: Sep. 10, 2009

(30) Foreign Application Priority Data
Jul. 24, 2006 (GB) 0614654.2

(51) Int. Cl. A61N 5/06

(52) U.S. Cl. 607/90

(57) ABSTRACT

A replaceable cartridge (10) for an intense pulsed light (IPL) device (100), comprises a discrete housing (11) allowing replacement of the cartridge in the IPL device, the housing including: a fluid inlet (12) for cooling fluid, a fluid outlet (14) for said fluid and a fluid cooling path (16) in fluid communication with said inlet and outlet; an electric lamp (20) within the housing adjacent said cooling path; and an electrical connection (18) for connecting to an electrical supply (118), said connection being in electrical communication with said lamp.

This invention relates to apparatus suitable for treating the skin area of a person by means of intense pulsed light (IPL). In particular, but not exclusively, the invention relates to a replaceable cartridge for such apparatus.

IPL devices are known for treating the skin, for example for removing hair, for photo rejuvenation and for treating skin disorders.

Conventionally, a gas discharge lamp is used to produce a pulse of intense broadband light. The intense light is pulsed at intervals to suit the treatment. The generation of this light produces excess heat energy, which must be removed from the area of the lamp. It is known commercially to remove heat from skin treatment devices by liquid cooling.

One problem with the system mentioned above is that the electrical supply used to power the intense light source must be completely isolated from the cooling circuit. This is particularly important when high voltages are employed. When the lamp needs to be replaced then, depending on the design, the cooling liquid can wet the electrical connections to the lamp or other electrical components.

Further, handling of the new lamp during changing, particularly when liquid is present will cause contamination of the lamp surface which in turn may result in premature failure of the lamp. Additionally, in known arrangements various screws and other components can be lost or incorrectly fitted when the lamp is changed. All of this results in the requirement for a skilled person to change the lamp.

Embodiments of the present invention address the problems mentioned above.

According to the present invention there is provided a replaceable cartridge for an intense pulsed light (IPL) device, the cartridge comprising a discrete housing allowing replacement of the cartridge in the IPL device, the housing including: a fluid inlet for cooling fluid, a fluid outlet for said fluid and a fluid cooling path in fluid communication with said inlet and outlet; an electric lamp within the housing adjacent said cooling path; and an electrical connection for connecting to an electrical supply, said connection being in electrical communication with said lamp.

Conveniently, the cartridge has a light transmissive window at least partially covering the lamp and optionally at least partially forming a wall for the fluid path.

Suitably, the lamp is located within a lamp cavity and the fluid path runs through the lamp cavity so that fluid flow takes place over the lamp and the window.

Conveniently, a further wall of the fluid path comprises a light reflector adjacent the lamp.

The invention extends to IPL device comprising a cartridge receiving area suitable for accepting the cartridge mentioned above, said area having at least a fluid supply adapted for supplying fluid to said inlet of the cartridge and a complementary electrical connection adapted for supplying electrical power or signals to said electrical connection of the cartridge.

Conveniently, the fluid supply has a valve for stopping or restricting fluid supply.

Conveniently the valve stops or restricts fluid supply automatically when the cartridge is disconnected from said cartridge receiving area.

Conveniently, the cartridge receiving area is located toward one end of an elongate flexible member.

More conveniently, the area is in the form of a socket or plug having a form complementary to the cartridge.

Optionally, the complementary electrical connection includes a data path for carrying data between the cartridge and the IPL device.

An embodiment of the invention will now be described, by way of example only, with reference to the following drawings, wherein:

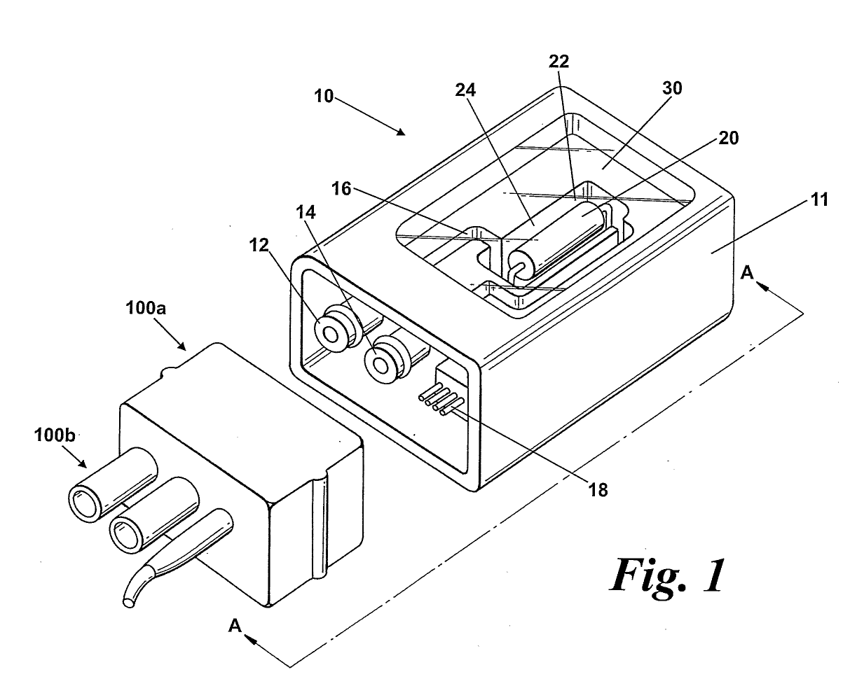

FIG. 1 shows a perspective. view of a cartridge and cartridge plug according to the invention, and

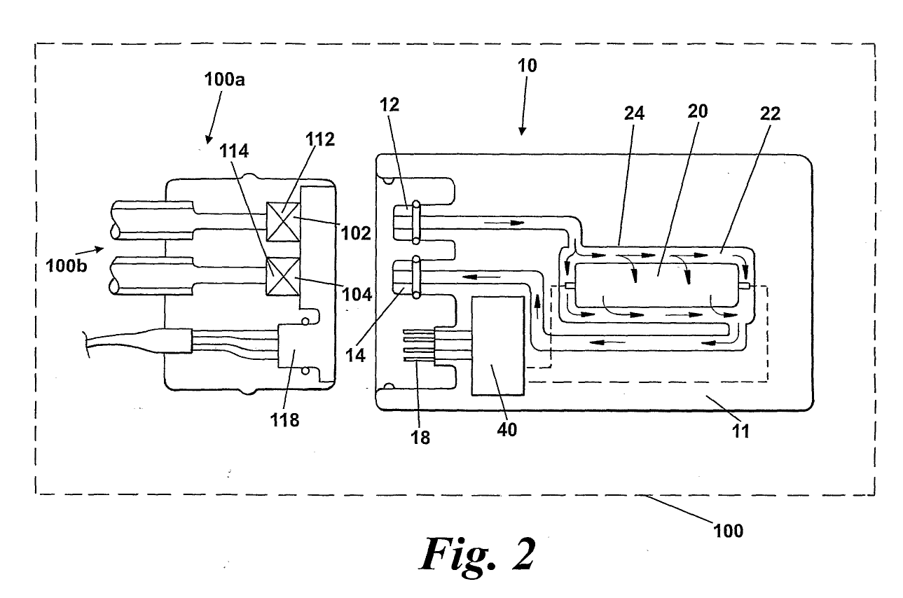

FIG. 2 shows a section in the plane “A-A” shown in FIG. 1.

Referring to FIGS. 1 & 2 a cartridge 10 is shown for use in IPL apparatus 100, shown schematically in broken outline in FIG. 2. The IPL apparatus 100 includes a cartridge supply plug 100a on the end of an elongate supply line 100b, as well as cartridge 10.

The cartridge 10 is a readily replaceable item which can be removed from the remaining IPL apparatus by unplugging from the plug 100a. In use the disconnection of the cartridge 10 from the plug 100a causes automatic closure of the open ends of the fluid supply 102 and return 104 (FIG. 2) at the plug 100a.

Cartridge 10 is a snug pushfit into plug 100a and includes a cooling fluid inlet 12, a cooling fluid outlet 14, and a cooling fluid path 16 in fluid communication with the inlet 12 and outlet 14. In this embodiment the fluid is water, but any suitable liquid or gas may be used. The cooling fluid flows in the direction of the arrows shown in FIG. 2. The cartridge includes an electrical connector 18 in the form of multiple pins. These pins provide at least a power supply and trigger signal. In this embodiment connector pins are provided for power, a trigger signal, and communication with remaining parts of the IPL device, as well as a common line.

Plug 100a has complementary formations: 112 to accept inlet 12; 114 to accept outlet 14 and; 118 to make electrical contact with the pins of the electrical connection 18. Each of these complementary formations is connected to the IPL apparatus by the supply line 100b, which in this case is flexible to allow manipulation of the cartridge 10 or any further parts of the IPL apparatus in which the cartridge may be removably fitted.

The cartridge 10 has a housing 11, which includes a gas discharge lamp 20 within a chamber 22. The faces 24 of the walls of the chamber 22 may have a reflective coating or alternatively a ceramic or similar diffuse reflector may be employed. The housing has a light transmissive window in the form of a glass face 30, which seals the chamber 22 and fluid path 16 against leaks. In use the lamp is surrounded by circulating cooling fluid.

As well as supplying power to the lamp and its trigger function, all controlled by the IPL device, the connector 18 is electrically connected to electronic device 40. The electronic device 40 provides secure communication to the remaining parts of the IPL apparatus 100 to identify the cartridge type and count the number of light pulses fired by the lamp. This aids safe operation of the cartridge over its life and enables the supplier to control the number of pulses that may be fired from a cartridge.

The light from lamp 20 propagates from the lamp through the glass face 30 and toward the skin of a person being treated, usually by means of an optical coupler (not shown).

This embodiment of the invention allows for an easily replaceable cartridge containing, in this instance, a discharge lamp 20 and a cooling circuit. This embodiment of the invention therefore overcomes the problem of changing the discharge, lamp 20 associated with conventional IPL apparatus.

Modifications, variants, alternatives, substitutions etc may be made to the embodiment mentioned above without departing from the scope of the invention. For example, it will be possible to change the shape of the cartridge and plug shown and their internal arrangements. In particular, the shape of the cartridge can be changed so that it forms a portable handheld IPL applicator head. A plug 100a has been shown, however, it will be readily apparent that this plug could be in the form of a socket into which a cartridge 10 is inserted or any suitable shape.

Incorporation of the elements mentioned in the embodiment above, into a cartridge of an IPL device has the following further advantages:

The end user, for example a beautician, is able to change the cartridge without having to send the IPL device away for attention or waiting for a visit from an engineer, thus improving users' satisfaction in the device;
The end user can simply change the cartridge if it fails during treatment, rather than cancel the treatment;
Distributors can spend less time servicing the IPL device and will have fewer resources invested in stocks of spare parts;
The cartridge will deter third parties from attempting a repair of the device;
The cartridge can be calibrated at the factory prior to shipment so that there is no need for end user adjustment; and
The IPL device can be programmed such that only a predetermined number of cartridges can be changed before a device service is required, thus reducing the likelihood of general failure of the device.

1. A replaceable cartridge for an intense pulsed light (IPL) device, the cartridge comprising a discrete housing allowing replacement of the cartridge in the IPL, device, the housing including: a fluid inlet for cooling fluid, a fluid outlet for said fluid and a fluid cooling path in fluid communication with said inlet and outlet; an electric lamp within the housing adjacent said cooling path; and an electrical connection for connecting to an electrical supply, said connection being in electrical communication with said lamp.

2. A cartridge according to claim 1 wherein the cartridge has a light transmissive window at least partially covering the lamp.

3. A cartridge according to claim 2 wherein the window at least partially forms a wall for the fluid path.

4. A cartridge according to claim 1, wherein the lamp is located within a lamp cavity and the fluid path runs through the lamp cavity so that fluid flow takes place over the lamp and the window.

5. A cartridge according to claim 1, wherein a further wall for the fluid path comprises a light reflector adjacent the lamp.

6. An intense pulsed light (IPL) device comprising a cartridge receiving area suitable for accepting a cartridge according to any preceding claim, said area having at least a fluid supply adapted for supplying fluid to said inlet of the cartridge and a complementary electrical connection adapted for supplying electrical power or signals to said electrical connection of the cartridge.

7. An IPL device according to claim 6 wherein the fluid supply has a valve for stopping or restricting fluid supply.

8. An IPL device according to claim 6 wherein the valve stops or restricts fluid supply automatically when the cartridge is disconnected from said cartridge receiving area.

9. An IPL device according to claim 6 wherein the cartridge receiving area is located toward one end of an elongate flexible member.

10. An IPL device according to claim 9 wherein the cartridge receiving area is in the form of a socket or plug having a form complementary to a corresponding end of the cartridge.

11. An IPL device according to claim 10 wherein the complementary electrical connection includes a data path for carrying data between the cartridge and the IPL device.

12. A cartridge according to claim 2, wherein the lamp is located within a lamp cavity and the fluid path runs through the lamp cavity so that fluid flow takes place over the lamp and the window.

13. A cartridge according to claim 3, wherein the lamp is located within a lamp cavity and the fluid path runs through the lamp cavity so that fluid flow takes place over the lamp and the window.

14. A cartridge according to claim 2, wherein a further wall for the fluid path comprises a light reflector adjacent the lamp.

15. A cartridge according to claim 3, wherein a further wall for the fluid path comprises a light reflector adjacent the lamp.

16. A cartridge according to claim 4, wherein a further wall for the fluid path comprises a light reflector adjacent the lamp.

17. An IPL device according to claim 7 wherein the cartridge receiving area is located toward one end of an elongate flexible member.

18. An IPL device according to claim 8 wherein the cartridge receiving area is located toward one end of an elongate flexible member.

19. An IPL device according to claim 17, wherein the cartridge receiving area is in the form of a socket or plug having a form complementary to a corresponding end of the cartridge.

20. An IPL device according to claim 18, wherein the cartridge receiving area is in the form of a socket or plug having a form complementary to a corresponding end of the cartridge.
